(19) United States
(12) Patent Application Publication
(10) Pub. No.: US 2022/0000396 A1
(43) Pub. Date: Jan. 6, 2022

(54) LIVING BODY DETECTION DEVICE

(71) Applicant: National University Corporation Yamagata University, Yamagata (JP)

(72) Inventors: Daisuke KUMAKI, Yonezawa-Shi (JP); Hiroto SATO, Yonezawa-shi (JP); Shizuo TOKITO, Yonezawa-shi (JP)

(73) Assignee: National University Corporation Yamagata University, Yamagata (JP)

(21) Appl. No.: 17/480,483

(22) Filed: Sep. 21, 2021

Related U.S. Application Data
(63) Continuation of application No. PCT/JP2020/014015, filed on Mar. 27, 2020.

(30) Foreign Application Priority Data
Mar. 28, 2019 (JP) 2019-064311

(51) Int. Cl. A61B 5/11; A61B 5/0205; G16H 50/70; G16H 40/67; G01L 1/16; G08B 21/02

(52) U.S. Cl. CPC A61B 5/1115; A61B 5/0205; G16H 50/70; A61B 5/024; G01L 1/16; G08B 21/02; G16H 40/67

(57) ABSTRACT

A living body detection device configured to detect a presence of a living body in a predetermined location. The living body detection device includes a piezoelectric element configured to detect a pressure change in the predetermined location, and a processor configured to: calculate multiple pieces of living body information, based on the pressure change detected by the piezoelectric element; calculate a composite index which compositively indicates that the multiple pieces of living body information are caused by the living body, based on the calculated multiple pieces of living body information; and determine whether there is or not the living body in the predetermined location, based on the calculated composite index.

### CROSS-REFERLNCE TO RELATED APPLICATIONS

The present application is a continuation application. of PCT International Application No. PCT/JP2020/014015 filed on Mar. 27, 2020 and claims priority from Japanese Patent Application No. 2019-064311 filed on Mar. 28, 2019, and the entire contents of which are hereby incorporated by reference.

### TECHNICAL FIELD

The present invention relates to a living body detection device, more specifically to a living body detection device configured to detect a living body by a pressure change in a predetermined location.

### BACKGROUND ART

Conventionally, a living body detection device configured to detect a pressure change in a predetermined location to detect whether there is a living body in the location has been in practical use. For example, by disposing the living body detection device on a bed in a hospital, it is possible to detect whether a patient leaves the bed based on the pressure change, and therefore to easily know the behavior of the patient. Here, there is demand to precisely detect the patient leaving the bed.

Here, as a technology of precisely detecting the patient leaving the bed, there has been proposed an in-bed detection device configured to be able to respond to a change in the initial value of the compressive load and reliably detect that the compressive load reaches a set value, for example, in Japanese Patent Application Laid-Open No. H10-9976. The entire contents of this disclosure are hereby incorporated by reference. This in-bad detection device detects that the position of a shaft member is changed up to the set value of the compressive load being applied, and therefore can easily respond to even when the initial value of the compressive load is changed, and consequently detect the motion of the patient leaving the bed in details.

### SUMMARY OF INVENTION

An aspect of the present invention provides a living body detection device configured to detect a presence of a living body in a predetermined location. The living body detection device includes a piezoelectric element configured to detect a pressure change in the predetermined location, and a processor configured to: calculate multiple pieces of living body information, based on the pressure change detected by the piezoelectric element; calculate a composite index which compositively indicates that the multiple pieces of living body information are caused by the living body, based on the calculated multiple pieces of living body information; and determine whether there is or not the living body in the predetermined location, based on the calculated composite index.

### BRIEF DESCRIPTION OF DRAWINGS

FIG. 1 is a block diagram illustrating the configuration of a living body detection device according to Embodiment 1 of the invention;

FIG. 2 illustrates a state where a detector is disposed on a bed;

FIG. 3 conceptually illustrates a probability density function stored in a memory;

FIG. 4 illustrates essential parts of the living body detection device according to Embodiment 2;

FIG. 5 illustrates essential parts of the living body detection device according to Embodiment 3; and

FIG. 6 illustrates essential parts of the living body detection device according to Embodiment 4.

### DESCRIPTION OF EMBODIMENTS

The in-bed detection device disclosed in Japanese Patent Application Laid-Open No. H10-9976 detects the presence of a living body only by the compressive load, and therefore might erroneously detect the presence of a living body, for example, when baggage is put on the bed. The present invention has been made to solve the above-described conventional problem, and it is therefore an object of the present invention to provide a living body detection device configured to precisely detect the presence of a living body in a predetermined location.

Hereinafter, the embodiments of the invention will be described with reference to the accompanying drawings.

### Embodiment 1

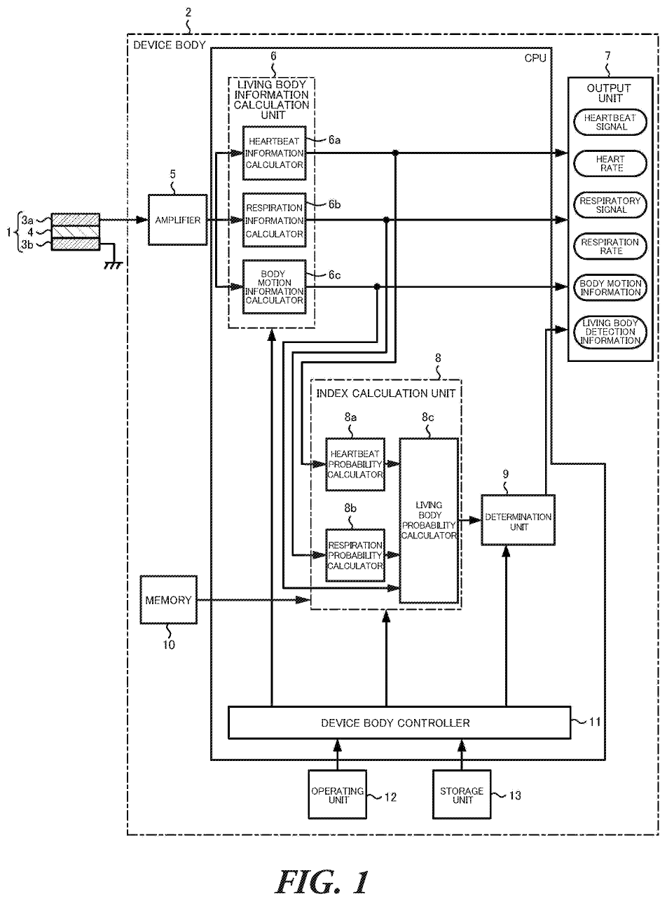

FIG. 1 illustrates the configuration of a living body detection device according to Embodiment 1. This living body detection device includes a detector 1 and a device body 2.

The detector 1 is configured to detect the pressure in a predetermined location and generate a voltage corresponding to the pressure. Specific examples of the detector 1 include a piezoelectric element. An embodiment of the detector 1 includes a pair of electrodes 3a and 3b and a ferroelectric layer 4. The electrodes 3a and 3b are electrically connected to the ferroelectric layer 4, and made of, for example, a conductive material such as a metallic material and an organic conductive material. The electrode 3a is connected to the device body 2, and the electrode 3b is grounded. The ferroelectric layer 4 is made of a ferroelectric material. The ferroelectric layer 4 may be made of, for example, a polyvinylidene fluoride (PVDF), and a poly(vinylidene-trifluoroethylene) copolymer (P(VDF-TrFE)).

The device body 2 includes an amplifier 5, and an output unit 7 is connected to the amplifier 5 via a living body information calculation unit 6. In addition, the living body information calculation unit 6 is connected to an index calculation unit 8, and the index calculation unit 8 is connected to the output unit 7 via a determination unit 9. A memory 10 is connected to the index calculation unit 8. Moreover, a device body controller 11 is connected to the living body information calculation unit 6, the index calculation unit 8 and the determination unit 9. An operating unit 12 and a storage unit 13 are connected to the device body controller 11.

The amplifier 5 is connected to the electrode 3a of the detector 1, and configured to amplify an electric signal from the detector 1. Specific examples of the amplifier 5 include an amplifier circuit. The living body information calculation unit 6 is configured to calculate multiple pieces of living body information based on the pressure change detected by the detector 1, and includes a heartbeat information calculator 6a, a respiration information calculator 6b, and a body motion information calculator 6c which are connected to the amplifier 5.

The heartbeat information calculator 6a calculates a heartbeat signal indicating the variable waveform of a heartbeat, and a heart rate indicating the number of heartbeats per minute, based on the electric signal amplified by the amplifier 5. To be more specific, the heartbeat information calculator 6a extracts a predetermined frequency band, for example, a band of 2 Hz to 10 Hz as a heartbeat signal, from the electric signal. In addition, the heartbeat information calculator 6a calculates the heart rate from the extracted heartbeat signal.

The respiration information calculator 6b calculates a respiratory signal indicating the variable waveform of respiration, and a respiration rate indicating the number of respiration per minute, based on the electric signal amplified by the amplifier 5. To be more specific, the respiration information calculator 6b extracts a predetermined frequency band, for example, a band of 0.1 Hz to 2 Hz as a respiratory signal, from the electric signal. In addition, the respiration information calculator 6b calculates the respiration rate from the extracted respiratory signal. The body motion information calculator 6c calculates body motion information indicating the body motion of a living body, based on the electric signal amplified by the amplifier 5. For example, whether or not there is a body motion is calculated as the body motion information. To be more specific, the body motion information calculator 6c determines that there is a body motion when the intensity of the electric signal is higher than a predetermined value, and, determines that there is no body motion when the intensity of the electric signal is equal to or lower than the predetermined value.

The memory 10 previously stores the probability density function corresponding to the heart rate, and the probability density function corresponding to the respiration rate. The probability density function of the heart rate is previously calculated based on the heart rates obtained from a plurality of different living bodies. Likewise, the probability density function of the respiration rate is previously calculated based on the respiration rates obtained from a plurality of different living bodies. Specific examples of the memory 10 include a RAM, a RPM, a hard disk drive, and a solid state drive.

The index calculation unit 8 includes a heartbeat probability calculator 8a connected to the heartbeat information calculator 6a, a respiration probability calculator 8b connected to the respiration information calculator 6b, and a living body probability calculator 8c connected to the heartbeat probability calculator 8a, the respiration probability calculator 8b and the body motion information calculator 6c. The heartbeat probability calculator 8a calculates the heartbeat probability indicating a probability that the heartbeat information detected by the detector 1 is caused by the heartbeat of the living body, from the probability density function of the heart rate stored in the memory 10, based on the heart rate calculated by the heart rate information calculator 6a. The respiration probability calculator 8b calculates the respiration probability indicating a probability that the respiration information detected by the detector 1 is caused by the respiration of the living body, from the probability density function of the respiration rate stored in the memory 10, based on the respiration rate calculated by the respiration rate information calculator 6b.

The living body probability calculator 8c is configured to calculate a composite index which compositively indicates that the heart rate, the respiration rate, and the body motion are caused by the living body, based on the heart rate, the respiration rate and the body motion calculated by the living body information calculation unit 6. The living body probability calculator 8c calculates the composite index by combining the heartbeat probability calculated by the heartbeat probability calculator 8a and the respiration probability calculated by the respiration probability calculator 8b by multiplication. In this case, the living body probability calculator 8c calculates the composite index when the body motion information calculator 6c determines that there is no body motion, and reduces the composite index or stops calculating the composite index when the body motion information calculator 6c determines that there is a body motion. By this means, the composite index is calculated, which is compounded of the heartbeat probability and the respiration probability, and also the body motion information. Here, the higher the value of the composite index, the higher the possibility that multiple pieces of living body information are caused by the living body.

The determination unit 9 determines whether there is a living body in the predetermined location based on the composite index calculated by the 1iv:ing body probability calculator 8c, and generates living body detection information indicating the result of the determination. The output unit 7 is configured to output the multiple pieces of living body information calculated by the living body information calculation unit 6 and the living body detection information generated by the determination unit 9 to the outside, and is connected to the heartbeat information calculator 6a, the respiration information calculator 6b, the body motion information calculator 6c, and the determination unit 9. Examples of the output unit 7 include a saving unit configured to sequentially save the living body information and the living body detection information to output them, a display unit configured to display the living body information and the living body detection information, an announcement unit configured to announce the contents of the living body information and the living body detection. information to the outside, and an communication unit configured to transmit the living body information and the living body detection information to an external device. Specific examples of the output unit 7 include a liquid crystal display, a speaker, an LED lamp, and a wired or wireless communication device.

The device body controller 11 controls each of the units of the living body detection device, based on an operating program stored in the storage unit 13 and commands inputted from the operating unit 12 by an operator. The operating unit 12 is configured to be used by the operator for input operation, and may be formed of, for example, a keyboard, a mouse, a track ball and a touch panel. Here, the operating unit 12 may not be disposed in the device body 2. For example, an operating signal of the operator may be inputted from an operating unit of an external device connected to the device body 2.

The storage unit 13 is configured to store the operating program and so forth, and may be a recording medium such as a flash ROM, an SD memory card, a micro SD memory card, an EEPROM, a hard disk, a flexible disk, an MO, an MT, a RAM, a CD-ROM, and a DVD-ROM. Here, the living body information calculation unit 6, the index calculation unit 8, the determination unit 9 and the device body controller 11 are configured by a CPU (Central processing unit: processor) and the operating program which causes the CPU to perform various processing, but they may be configured by a digital circuit.

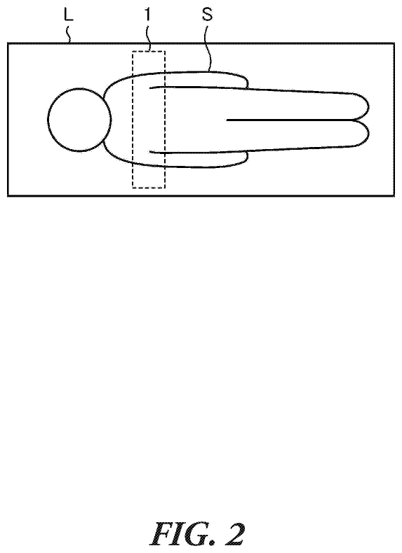

Next, the operation of Embodiment 1 will be described. First, as illustrated in FIG. 2, the detector 1 is disposed on a predetermined location, for example, a bed L such as a sleeping bed on which a living body S lies down. The detector 1 is formed to extend in the width direction of the bed L, and configured to be able to sequentially detect a pressure change from the living body S lying down on the bed L. In response to this pressure change, the detector 1 outputs an electric signal to each of the heartbeat information calculator 6a, the respiration information calculator 6b, and the body motion information calculator 6c via the amplifier 5 as illustrated in FIG. 1.

When receiving the electric signal of the pressure change, the heartbeat information calculator 6a extracts a predetermined frequency band from the electric signal, and generates a heartbeat signal. In addition, the heartbeat information calculator 6a calculates a heart rate from the generated heartbeat signal. Then, the heartbeat information calculator 6a outputs the heartbeat signal and the heart rate to the output unit 7, and also outputs the heart rate to the heartbeat probability calculator 8a.

Likewise, when receiving the electric signal of the pressure change, the respiration information calculator 6b generates a respiratory signal from the electric signal, and calculates a respiration rate. Then, the respiration information calculator 6b outputs the respiratory signal and the respiration rate to the output unit 7, and also outputs the respiration rate to the respiration probability calculator 8b. When receiving the electric signal of the pressure change, the body motion information calculator 6c determines that there is a body motion in the case where the intensity of the electric signal is higher than a predetermined value, and determines that there is no body motion in the case where the intensity is equal to or lower than the predetermined value. Then, the body motion information calculator 6c outputs the information indicating whether there is or not a body motion to each of the output unit 7 and the living body probability calculator 8c.

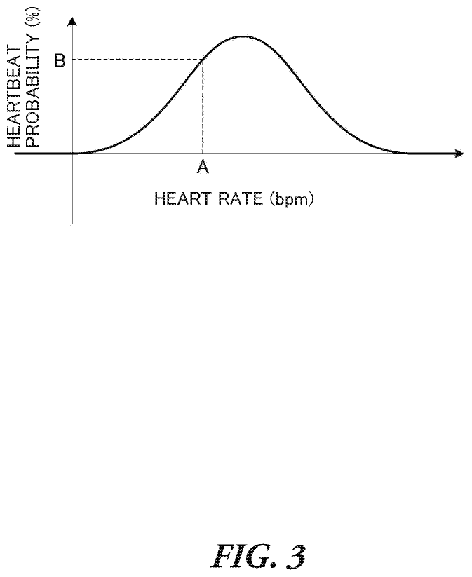

When receiving the heart rate, the heartbeat probability calculator 8a calculates a heartbeat probability indicating that the heartbeat information detected by the detector 1 is caused by the heartbeat of the living body S, based on the heart rate. Here, as illustrated in FIG. 3, the probability density function calculated based on the heart rates of a plurality of living bodies is stored in the memory 10 in advance. Referring to the probability density function of the heart rates stored in the memory 10, the heartbeat probability calculator 8a calculates heartbeat probability B of heart rate A calculated by the heartbeat information calculator 6a. The calculated heartbeat probability B is outputted from the heartbeat probability calculator 8a to the living body probability calculator 8c.

Likewise, when receiving the respiration rate, the respiration probability calculator 8b calculates a respiration probability indicating that the respiration information detected by the detector 1 is caused by the respiration of the living body S, based on the respiration rate. Here, the probability density function calculated based on the respiration rates of a plurality of living bodies is stored in the memory 10 in advance. Referring to the probability density function of the respiration rates stored in the memory 10, the respiration probability calculator 8b calculates a respiration probability of the respiration rate calculated by the respiration information calculator 6b. The calculated respiration probability is outputted from the respiration probability calculator 8b to the living body probability calculator 8c.

In this way, the probability density functions calculated in advance are stored in the memory 10, and therefore the heartbeat probability calculator 8a and the respiration probability calculator 8b can easily calculate the heartbeat probability B and the respiration probability based on the probability density functions.

When receiving the heartbeat probability B calculated by the heartbeat probability calculator 8a, the respiration probability, calculated by the respiration probability calculator 8b, and the body motion information calculated by the body motion information calculator 8c, the living body probability calculator 8c calculates a composite index compounded of the heartbeat probability B, the respiration probability, and the body motion information. To be more specific, the living body probability calculator 8c calculates the composite index obtained by multiplying the heartbeat probability B and the respiration probability together, when the body motion information indicates that there is no body motion of the living body S. On the other hand, when the body motion information indicates that there is a body motion of the living body S, the living body probability calculator 8c stops calculating the composite index. Here, the living body probability calculator 8c may calculate a weighted composite index which is weighted based on, for example, the reliability and the validity of the heartbeat probability B and the respiration probability. The calculated composite index is outputted from the living body probability calculator 8c to the determination unit 9.

When receiving the composite index from the living body probability calculator 8c, the determination unit 9 determines whether there is or not the living body S on the bed L based on the composite index. That is, the determination unit 9 determines that there is the living body S on the bed L when the composite index is higher than a predetermined threshold, and determines that there is not the living body S on the bed L when the composite index is equal to or lower than the predetermined threshold. The determination unit 9 outputs the result of the determination as living body detection information to the output unit 7.

In this way, the determination unit 9 determines the presence of the living body S based on the composite index compounded of the heartbeat probability B, the respiration probability, and the body motion information, and therefore can make a precise determination compared to when a determination is made based on one index. In addition, the determination unit 9 can determine the presence of the living body S based on the value of the composite index which continuously changes, and therefore can make a precise determination compared to when a plurality of indexes are determined individually. Moreover, the determination unit 9 makes a determination by the composite index calculated based on the heart rate and the respiration rate which are continually acquired, and therefore can precisely determine the presence of the living body S, compared to when a determination is made based on the body motion information obtained at a time the living body S moves. Furthermore, the determination unit 9 makes a determination based on the composite index compounded of the heart rate and the respiration rate, and also the body motion information having a different property, and therefore can more precisely determine the, presence of the living body S.

The living body probability calculator 8c stops calculating the composite index when the body motion information calculator 6c calculates that there is a body motion of the living body S. Generally, when there is a body motion of the living body S, the heart rate and the respiration rate tend to indicate abnormal values which are remarkably different from normal values. Therefore, by stopping the calculation of the composite index, it is possible to prevent the determination unit 9 from erroneously determining the presence of the living body S based on the abnormal values. Here, it is preferred that, while stopping the calculation of the composite index based on the body motion information from the body motion information calculator 6c, the living body probability calculator 8c outputs the composite index just before the stop of the calculation to the determination unit 9. By this means, it is possible to prevent the determination unit 9 from erroneously determining that there is not the living body S when a body motion of the living body S occurs, and therefore to reliably determine the presence of the living body S. Next, when the body motion information calculator 6c calculates that there is no body motion of the living body S, the living body probability calculator 8c resumes the calculation of the composite index, and outputs the calculated new composite index to the determination unit 9.

When receiving the heartbeat signal, the heart rate, the respiratory signal, the respiration rate, and the body motion information from the living body information calculation unit 6, and also receiving the living body detection information from the determination unit 9, the output unit 7 outputs these pieces of information, or saves these pieces of information to output them. In this way, the output unit 7 outputs the living body detection information as well as the heartbeat signal, the heart rate, the respiratory signal, the respiration rate, and the body motion information, and consequently the user can easily recognize the reliability of the living body information.

According to the present embodiment, the determination unit 9 determines whether there is or not the living body S on the bed L based on the composite index compounded of the multiple pieces of living body information, and therefore it is possible to precisely detect the presence of the living body S.

### Embodiment 2

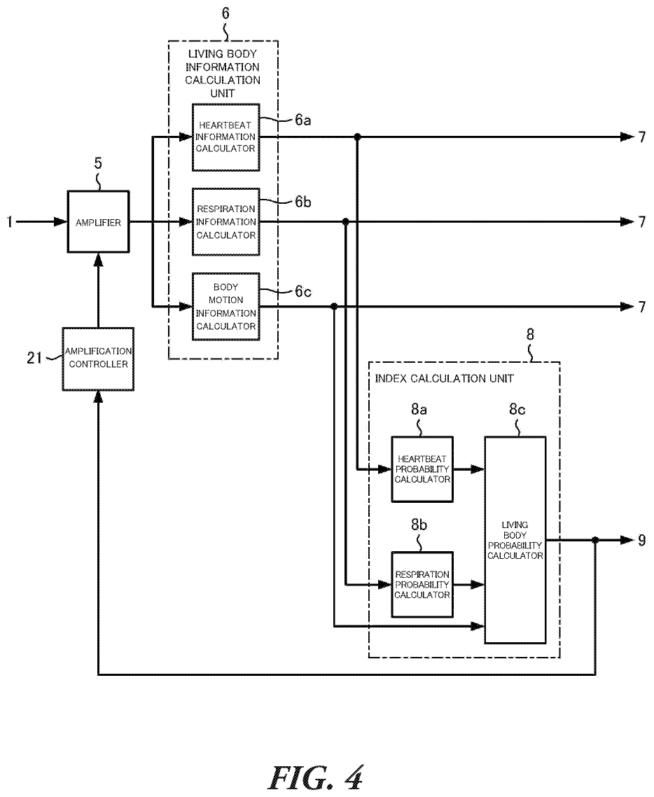

With the above-described Embodiment 1, the amplifier 5 amplifies the electric signal outputted from the detector 1 with a predetermined amplification factor. Here, the amplification factor for the electric signal can be optimized based on the composite index calculated by the index calculation unit 8. For example, with Embodiment 1, an amplification controller 21 may be newly disposed as illustrated in FIG. 4.

The amplification controller 21 is connected to the living body probability calculator 8c of the index calculation unit 8 and the amplifier 5, and configured to control the amplification of the amplifier 5 based on the composite index calculated by the living body probability calculator 8c. To be more specific, the amplification controller 21 changes the amplification factor of the amplifier 5 for the electric signal outputted from the detector 1 to maximize the composite index calculated by the index calculation unit 8 for the electric signal.

With this configuration, when receiving the composite index calculated by the living body probability calculator 8c, the amplification controller 21 changes, for example, increases the amplification factor of the amplifier 5 from the previous first amplification factor by a predetermined value. Then, the amplifier 5 amplifies the electric signal outputted from the detector 1 with the resultant second amplification factor; the living body information calculation unit 6 calculates multiple pieces of living body information; and the index calculation unit 8 calculates the composite index. Next, when the composite index calculated by the index calculation unit $ is higher than the previous composite index, the amplification controller 21 increases the amplification factor of the amplifier 5 to a value higher than the second amplification factor. On the other hand, when the composite index calculated by the index calculation unit 8 is lower than the previous composite index, the amplification controller 21 reduces the amplification factor of the amplifier 5 to a value lower than the second amplification factor. In this way, the amplification controller 21 can adjust the amplification factor of the amplifier 5 to maximize the composite index, and therefore the living body information calculation unit 6 can appropriately calculate the multiple pieces of living body information.

Here, it is preferred that the amplification controller 21 optimizes the amplification factor of the amplifier 5 just after the body motion information calculator 6c determines that the body motion of the living body S stops, that is, just after the determination that there is a body motion is changed to the determination that there is no body motion. Generally, when a body motion of the living body S occurs, the position at which the living body S contacts the detector 1 may be changed, and therefore the electric signal outputted from the detector 1 may be affected. The amplification controller 21 therefore optimizes the amplification factor of the amplifier 5 just after the body motion information calculator 6c determines that the body motion of the living body S stops. By this means, it is possible to prevent the multiple pieces of living body information from being varied due to the body motion of the living body S.

In addition, the amplification controller 21 may control the amplifier 5 to maximize the composite index based on the heartbeat probability and the respiration probability. When receiving the composite index, the heartbeat probability, and the respiration probability from the index calculation unit 8, the amplification controller 21 compares the heartbeat probability and the respiration probability. Here, for example, when the heartbeat probability is small, the amplification controller 21 changes the amplification factor of the amplifier 5 to maximize the heartbeat probability. As described above, the amplification controller 21 controls the amplification factor of the amplifier 5 based on the heartbeat probability and the respiration probability, and therefore can effectively maximize the composite index.

According to the present embodiment, the amplification controller 21 changes the amplification factor of the amplifier 5 for the electric signal outputted from the detector 1 to maximize the composite index calculated by the index calculation unit 8 for the electric signal. By this means, the living body information calculation unit 6 can appropriately calculate multiple pieces of living body information, and the determination unit 9 can more precisely determine the presence of the living body S.

### Embodiment 3

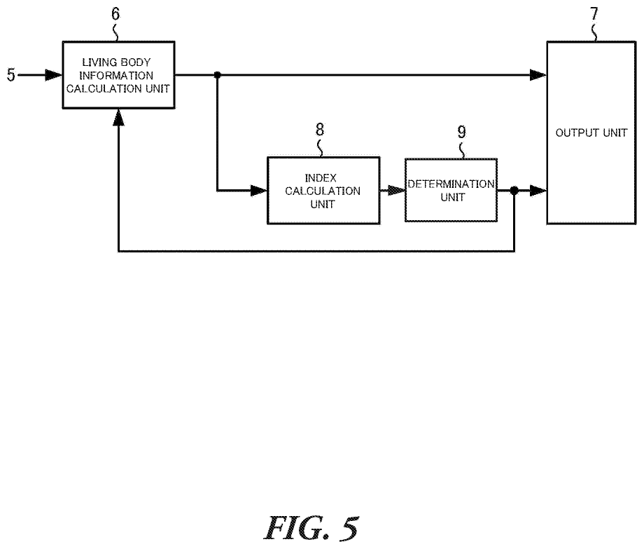

With the above-described. Embodiments 1 and 2, the living body information calculation unit 6 can control the output of multiple pieces of living body information to the output unit 7, based on the result of the determination of the determination unit 9. For example, as illustrated in FIG. 5, the determination unit 9 may be connected to the living body information calculation unit 6.

With this configuration, the determination unit 9 outputs the result of the determination to the living body information calculation unit 6 as well as the output unit 7 When the determination unit 9 determines that there is the living body S, the living body information calculation unit 6 outputs the calculated heartbeat signal, heart rate, respiratory signal, respiration rate, and body motion information to the output unit 7. On the other hand, when the determination unit 9 determines that there is not the living body S, the living body information calculation unit 6 stops outputting the calculated heartbeat signal, heart rate, respiratory signal, respiration rate, and body motion information to the output unit 7. In this way, the living body information calculation unit 6 stops outputting the multiple pieces of living body information to the output unit 7. By this means, for example, the user can recognize only reliable living body information, and therefore make an appropriate determination in diagnosis and so forth.

Here, when the determination unit 9 determines that there is not the living body S, it is preferred that the living body information calculation unit 6 outputs the living body information to the output unit 7 just before the stop of the output to the output unit 7, instead of the living body information sequentially calculated.

According to the present embodiment, when the determination unit 9 determines that there is not the living body S, the living body information calculation unit 6 stops outputting the multiple pieces of living body information to the output unit 7. By this means, it is possible to allow the output unit 7 to output reliable living body information.

### Embodiment 4

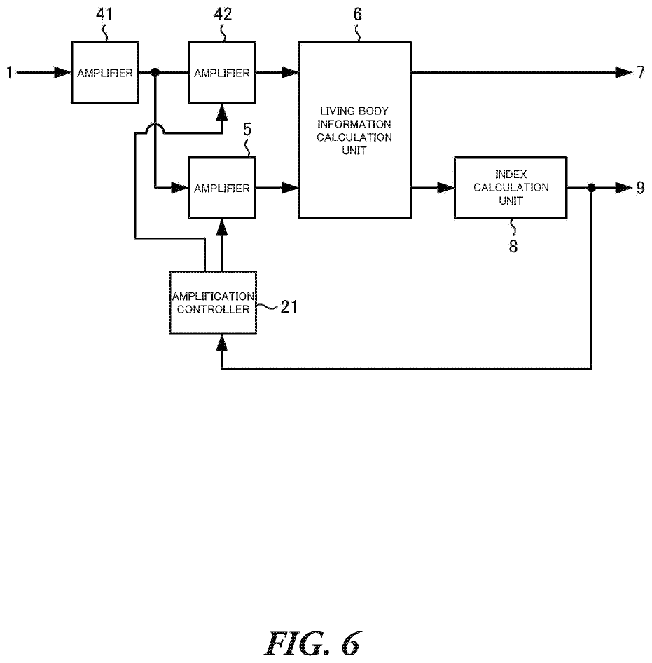

With the above-described Embodiments 1 to 3, the living body information calculation unit 6 inputs one electric signal amplified by the amplifier 5. However, the living body information calculation unit 6 may input a plurality of electric signals amplified by a plurality of amplifiers. For example, with Embodiment 2, an amplifier 41 may be newly disposed between the detector 1 and the amplifier 5, and an amplifier 42 may be newly disposed between the amplifier 41 and the living body information calculation unit 6, as illustrated in FIG. 6.

The amplifier 41 is configured to amplify the electric signal outputted from the detector 1 with a predetermined amplification factor. The amplifier 42 is connected to the amplification controller 21, and configured to amplify the electric signal outputted from the amplifier 41 under the control of the amplification controller 21. Here, the electric signal outputted from the amplifier 41 is also outputted to the amplifier 5. Then, the amplifier 5 amplifies this electric signal with a changed amplification factor under the control of the amplification controller 21 to maximize the composite index calculated by the index calculation unit 8.

With this configuration, the electric signal amplified by the amplifier 42 via the amplifier 41 is inputted to the living body information calculation unit 6, while the electric signal amplified by the amplifier 5 via the amplifier 41 is inputted to the living body information calculation unit 6. The living body information calculation unit 6 calculates the living body information based on the electric signal amplified by the amplifier 42, and outputs this living body information to the output unit 7. In addition, the living body information calculation unit 6 calculates the living body information based on the electric signal amplified by the amplifier 5, and outputs this living body information to the index calculation unit 8. Next, the index calculation unit 8 calculates the composite index based on the living body information calculated by the living body information calculation unit 6. Then, the amplification factor of the amplifier 5 is changed under the control of the amplification controller 21 to maximize the composite index calculated by the index calculation unit 8. After determining the amplification factor to maximize the composite index, the amplification controller 21 changes the amplification factor of the amplifier 42 to a value to maximize the composite index. Then, the living body information calculation unit 6 calculates the living body information based on the electric signal amplified by the amplifier 42 and outputs this living body information to the output unit 7. Meanwhile, the amplification controller 21 changes the amplification factor of the amplifier 5 to maximize the composite index calculated by the index calculation unit 8 in the same way.

In this way, the electric signal amplified by the amplifier 42 is used to calculate the living body information outputted to the output unit 7, and the electric signal amplified by the amplifier 5 is used to optimize the amplification factor. By this means, the living body information calculation unit 6 can calculate the living body information outputted to the output unit 7, based on the electric signal amplified with the amplification factor sequentially optimized, and output correct living body information to the output unit 7.

Here, the living body information calculation unit 6 may use the electric signal amplified by the amplifier 42 to calculate the heartbeat signal and the heart rate, and use the electric signal amplified by the amplifier 5 to calculate the respiratory signal and the respiration rate. According to the present embodiment, the electric signal amplified by the amplifier 42 and the electric signal amplified by the amplifier 5 are used for different purposes, and therefore it is possible to perform a wide variety of processing in the living body information calculation unit 6 and so forth.

Here, with the above-described Embodiments 1 to 4, the living body detection device is used for bedding where the detector 1 is disposed cm the bed L. However, this is by no means limiting as long as the detector 1 can detect a pressure change in a predetermined location.

For example, the living body detection device may be used for a seat on which the living body S sits and the detector 1 is disposed.

In addition, with the above-described Embodiments 1 to 4, the index calculation unit 8 calculates the composite index which compositively indicates that the heart rate, the respiration rate, and the body motion are caused by the living body. However, this is by no means limiting as long as the index calculation unit 8 can calculate the composite index which compositively indicates that the multiple pieces of living body information are caused by the living body.

Moreover, with the above-described Embodiments 1 to 4, the index calculation unit 8 calculates the composite index based on the probability density function. However, this is by no means limiting as long as the index calculation unit 8 can calculate the composite index which compositively indicates that, the multiple pieces of living body information calculated by the living body information calculation unit 6 are caused by the living body.

Furthermore, with the above-described Embodiments 1 to 4, the index calculation unit 8 calculates the composite index by calculating the heartbeat probability B and the respiration probability individually. However, this is by no means limiting as long as the index calculation unit 8 can calculate the composite index which compositively indicates that the multiple pieces of living body information calculated by the living body .information calculation unit 6 are caused by the living body. For example, a three-dimensional probability density function where X-axis represents heart rate; Y-axis represents respiration rate; and Z-axis represents living body probability may be calculated in advance and stored in the memory 10, and the composite index may be calculated at one time based on the three-dimensional probability density function. Here, the living body probability compositively indicates that the heart rate and the respiration rate are caused by the living body, and may be calculated, for example, by multiplying the heartbeat probability and the respiration probability together.

Furthermore, with the above-described Embodiments 1 to 4, the detector 1 detects a pressure change by using the ferroelectric layer 4. However, this is by no means limiting as long as the detector 1 can detect a pressure change in a predetermined location. A piezoelectric layer made of, for example, polylactic acid, polyurea, and a porous material may be disposed instead of the ferroelectric layer 4.

1. A living body detection device configured to detect a presence of a living body in a predetermined location, the living body detection device comprising:
a piezoelectric element configured to detect a pressure change in the predetermined location; and
a processor configured to:
calculate multiple pieces of living body information, based on the pressure change detected by the piezoelectric element;
calculate a composite index which compositively indicates that the multiple pieces of living body information are caused by the living body, based on the calculated multiple pieces of living body information; and
determine whether there is or not the living body in the predetermined location, based on the calculated composite index.

2. The living body detection device according to claim 1, further comprising a memory configured to store probability density functions indicating probabilities that the multiple pieces of living body information are caused by the living body, the probability density functions being stored to correspond to the multiple pieces of living body information,
wherein the processor calculates a probability that the multiple pieces of living body information are caused by the living body for each of the multiple pieces of living body information, from the probability density functions stored in the memory, based on the calculated multiple pieces of living body information, and calculates the composite index by combining the probabilities with each other.

3. The living body detection device according to claim 2, wherein the multiple pieces of living body information include a heart rate and a respiration rate.

4. The living body detection device according to claim 3, wherein:
the multiple pieces of living body information include body motion information indicating a body motion of the living body; and
the processor calculates the composite index when calculating that there is no body motion of the living body, and stops calculating the composite index when calculating that there is a body motion of the living body.

5. The living body detection device according to claim 2, further comprising an amplifier configured to amplify an electric signal outputted from the piezoelectric element in response to the pressure change, and output the electric signal to the living body information calculation unit,
wherein the processor controls amplification of the amplifier.

6. The living body detection device according to claim 5, wherein the processor changes an amplification factor for the electric signal outputted from the piezoelectric element to maximize the calculated composite index for the electric signal.

7. The living body detection device according to claim 2, further comprising an output unit configured to output the calculated multiple pieces of living body information outside,
wherein when determining that there is no living body, the processor stops outputting the multiple pieces of living body information to the output unit.

8. A method executed by a processor for detecting a presence of a living body in a predetermined location, the method comprising:
calculating multiple pieces of living body information, based on a pressure change in the predetermined location detected by a piezoelectric element;
calculating a composite index which compositively indicates that the multiple pieces of living body information are caused by the living body, based on the calculated multiple pieces of living body information; and
determining whether there is or not the living body in the predetermined location, based on the calculated composite index.

9. A non-transitory computer-readable medium having a program recorded thereon that causes a processor to execute a process comprising:
calculating multiple pieces of living body information, based on a pressure change in a predetermined location detected by a piezoelectric element;
calculating a composite index which compositively indicates that the multiple pieces of living body information are caused by a living body, based on the calculated multiple pieces of living body information; and
determining whether there is or not the living body in the predetermined location, based on the calculated composite index.
